(19) United States
(12) Patent Application Publication
(10) Pub. No.: US 2004/0222095 A1
(43) Pub. Date: Nov. 11, 2004

(54) Dna separation using linear polymer solutions with dimethyl sulfoxide

(76) Inventors: Barry L. Karger, Newton, MA (US); Lev Kotler, Lynn, MA (US); Hui He, Hillsborough, NJ (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/250,797

(22) PCT Filed: Jan. 14, 2002
(86) PCT No.: PCT/US02/01004

Related U.S. Application Data
(60) Provisional application No. 60/261,689, filed on Jan. 12, 2001.

(51) Int. Cl. G01L 1/20; C07K 1/26; C02F 1/469; G01N 27/26

(52) U.S. Cl. 204/451

(57) ABSTRACT

The invention is directed to a high throughput nucleic acid separation method using an improved uncrosslinked polymer separation matrix for increasing read length and separation speed, while maintaining accuracy, for, e.g., nucleic acid sequencing. The separation matrix of the invention includes a denaturant comprising dimethyl sulfoxide (DMSO). Preferably, the separation matrix may further comprise urea. Preferred matrix polymers include linear polyacrylamide, poly(ethylene oxide), hydroxyethyl cellulose, poly(dimethylacrylamide) and poly(vinylpyrrolidone).

### CROSS REFERENCE TO RELATED APPLICATIONS

This application claims the priority of U.S. Provisional Application No. 60/261,689 filed on Jan. 12, 2001, entitled DNA SEPARATION USING LINEAR POLYMER SOLUTIONS WITH DIMETHYL SULFOXIDE, the whole of which is hereby,incorporated by reference herein.

### STATEMENT REGARDING FEDERALLY SPONSORED RESEARCH OR DEVELOPMENT

Part of the work leading to this invention was carried out with United States Government support provided under a grant from the Department of Energy, Human Genome Project Grant No. DE-FG02-90ER-60985. Therefore, the U.S. Government has certain rights in this invention.

### BACKGROUND OF THE INVENTION

Over the past decade, capillary electrophoresis (CE) has rapidly developed (Dovichi; Dolnik; Heller; Ruiz-Martinez et al., 1993; Zhou et al.; Tan et al.) to become the method for sequencing nucleic acids, particularly for the Human Genome Project (Venter; Lander). Even with the accomplishment of this project goal, high-throughput sequencing technology will still be required to complete other important genomes, as well as for applications in genetic screening, SNP discovery and scoring, pharmacogenomics, etc. (Weitzman; Schmitz et al.; Heath et al.). These demands create the need for even higher throughput from sequencing instrumentation.

Commercial DNA sequencers generally employ a separation matrix of linear polyacrylamide (LPA) with 6-7M urea as a denaturant. The optimum run temperature for a matrix of this composition has been found to be in the range of 60-70° C. Thus, current automated DNA sequencers using LPA solutions are designed to maintain a temperature at this level throughout the run. Typically, the read length per capillary run is no longer than 600-800 bases. An optimum separation of 1000 bases with 97% accuracy has been achieved at 150 V/cm and a column temperature of 50° C. with a matrix containing 2% w/w of a high molecular mass LPA (Carrilho et al.). However, the run time for this separation was 80 minutes. Therefore, improved methods of achieving an increase in both read length and separation speed, particularly for commercial instruments, while maintaining or even improving sequencing accuracy are clearly desirable.

### BRIEF SUMMARY OF THE INVENTION

The invention is directed to the use of a robust uncrosslinked polymer separation matrix that incorporates dimethyl sulfoxide (DMSO) as the basic denaturant for, e.g., sequencing long fragments of DNA with precision in a relatively short turn-around time. A concentration of 1% to 25% v/w DMSO is preferred, and most preferably 5% v/w DMSO. The improved separation matrix of the invention may also include urea, up to a concentration of 7M, and most preferably 2-3M. As one embodiment, the separation matrix of the invention is useful in DNA sequencing, and the method of the invention demonstrates optimal DNA sequencing with a denaturant mixture of 5% v/w DMSO and 2M urea in the separation matrix. This combination produced a long read length with high accuracy at a column temperature of 70° C. in only 40 minutes. The read length can be well over 900 bases with 98.5% accuracy. This denaturing mixture may be considered as an alternative to 6-7M urea if a significant increase in speed in DNA sequencing with LPA solutions is desirable. In addition, the urea concentration in the denaturant mixture can be adjusted upward to resolve more difficult templates.

Accordingly, the invention includes a method of high throughput nucleic acid sequencing, said method comprising the steps of:

a) providing a nucleic acid sample to be sequenced;

b) carrying out nucleic acid sequencing reactions on said sample, thereby generating a product;

c) injecting an aliquot of said product into a separation device, said device comprising an uncrosslinked polymer matrix solution and a denaturant comprising dimethyl sulfoxide;

d) separating said product into component parts using said device; and

e) determining the sequence of nucleotides in said nucleic acid sample from the results of said separation step.

The separation matrix of the invention may be used for capillary electrophoresis, wherein the capillary electrophoretic device includes a capillary column, which may be part of a capillary array. Alternatively, the capillary electrophoretic device may comprise a microscale liquid handling substrate (microchip) having one or more channels integrally formed therein for conducting a liquid sample in the substrate. The uncrosslinked polymer of the invention may include, inter alia, linear polyacrylamide (LPA), poly(ethylene oxide) (PEO), hydroxyethyl cellulose (HEC), poly(dimethylacrylamide) and poly(vinylpyrrolidone).

In an embodiment of the invention, a separation matrix for nucleic acid electrophoretic analysis, e.g., separation or sequencing of nucleic acids, comprises an uncrosslinked polymer matrix solution and a denaturant comprising dimethyl sulfoxide. In one aspect, the denaturant further includes urea. In another aspect, the denaturant is at a concentration of 1% to 25% v/w dimethyl sulfoxide and 0.5M to 7M urea. In a further aspect, the uncrosslinked polymer is selected from the group consisting of linear polyacrylamide (LPA), poly(ethylene oxide) (PEO), hydroxyethyl cellulose (HEC), poly(dimethylacrylamide) and poly(vinylpyrrolidone).

Preferably, the separation matrix of the invention comprises an LPA polymer matrix solution and a denaturant further comprising 1% to 25% v/w dimethyl sulfoxide, where the separation temperature is at a temperature in the range of 60° C. to 80° C. More preferably, the separation matrix also includes 0.5M to 7M urea. Even more preferably, the LPA matrix solution includes a denaturant comprising 5% v/w dimethyl sulfoxide and 2-3M urea.

The invention also includes a general method for electrophoretic analysis of nucleic acids, wherein the method comprises the steps of:

a) providing a nucleic acid sample to be analyzed;

b) carrying out steps of a nucleic acid analytical method that produce a product to be separated into component parts;

c) injecting an aliquot of said product into a separation device, said device comprising a uncrosslinked polymer matrix solution and a denaturant comprising dimethyl sulfoxide;

d) separating said product into component parts using said device; and

e) determining the final results of said analytical method on said nucleic acid sample from the results of said separation step.

The product to be separated into component parts may include, inter alia, a mixed population of different length nucleic acids or single or double-stranded nucleic acids that can be subjected to mild denaturants so as to relax a portion of their three-dimensional structures.

In the general method of invention, the uncrosslinked polymer is selected from the group consisting of linear polyacrylamide, poly(ethylene oxide), hydroxyethyl cellulose, poly(dimethylacrylamide) and poly(vinylpyrrolidone). The nucleic acid analytical method prior to separation can include single strand conformational polymorphism (SSCP) determination, constant denaturant/capillary electrophoresis (CD/CE) and restriction fragment length polymorphism (RFLP) analysis.

An appropriate temperature for the methods of the invention is a function of the type of polymer used and the sample to be denatured and separated. Therefore, the separation step d) may be conducted at a temperature of 25° C. to 50° C., more preferably at 50° C. or higher, more preferably at 60° C. or higher, even more preferably at 70° C. or higher, preferably at 70° C. to 80° C., and more preferably at 80° C. to 90° C.

### BRIEF DESCRIPTION OF THE DRAWINGS

Other features and advantages of the invention will be apparent from the following description of the preferred embodiments thereof and from the claims, taken in conjunction with the accompanying figures, in which:

FIGS. 1A-1C show electropherograms of the sequencing of M13mp18 template using 2.5% w/w 5.6 MDa LPA matrix containing 6M (A), 5M(B), and 3.5M (C) urea at a temperature of 65° C. A compression site is marked by an arrow;

FIG. 2 shows a plot of electric current in a capillary filled with a separation matrix containing 2.5% w/w 5.6 MDa LPA: with 7M urea (&Ovalhollow;)(1), with no denaturant (▪)(2), and with 5% v/w DMSO (▾) (3). The current was measured at 200 V/cm, 10 minutes after the start of the run. The current in graph (2) was adjusted to the same scale as in graphs (1) and (3) by a factor of 0.69. For each experiment at a given temperature, a fresh portion of the separation matrix was pumped into the capillary;

FIGS. 3A-3E show electropherograms of sequencing through a compression motif (a triplet of C-terminated peaks marked with an arrow) with 2.5% w/w 5.6 MDa LPA at 70° C. and 200 V/cm. The matrix contained as denaturant: 5% v/w DMSO (A); mixtures of 5% w/v DMSO and urea at a concentration of 1M (B), 2M (C) and 3M (D); and 7M urea alone (E); and

FIG. 4 shows an electropherogram of sequencing having a read length of 976 bases with 98.5% accuracy in less than 40 minutes with 2.5% w/w 5.6. MDa LPA solution containing 5% v/w DMSO and 2M urea at 70° C. and 200 V/cm.

### DETAILED DESCRIPTION OF THE INVENTION

In accordance with the invention, separation speed during DNA sequencing by capillary electrophoresis (CE) can be increased significantly while generating a long read length in addition to high accuracy within a relatively short analysis time. A separation matrix solution of the invention comprises an uncrosslinked polymer and a denaturant. The uncrosslinked polymer includes, inter alia, linear polyacrylamide (LPA), poly(ethylene. oxide) (PEO), hydroxyethyl cellulose (HEC), poly(dimethylacrylamide) and poly(vinylpyrrolidone). Particularly, an improved linear polyacrylamide (LPA) matrix has been developed that uses dimethyl sulfoxide (DMSO) as a denaturant. The denaturant further comprises urea. An LPA matrix using 5% (v/w) dimethyl sulfoxide (DMSO) and 2M urea as a denaturant generates a long read length with 98.5% accuracy in less than 40 minutes.

In DNA sequencing by CE, the separation speed may be increased by several means, including using short columns, increasing column temperature and/or electric field strength, or changing the separation matrix/buffer composition. However, the improvement with such changes often comes at the expense of read length. In previous work, the combination of various parameters for separation of 1000 bases in roughly one hour at 60-70° C. was demonstrated (Zhou et al.; Salas-Solano et al.). Such a system was optimized for the defined long read length in the shortest separation time. Shortening the capillary length yielded faster separations but shorter read lengths, and an increase in the electric field strength had similar effect.

One of the means for further optimization of DNA sequencing by CE for faster speeds can be to lower the viscosity of the LPA solution. The solution viscosity of a separation matrix is mainly determined by polymer, e.g., LPA, properties (average molecular mass and its concentration in the solution) and the urea concentration. The LPA concentration and molecular mass have already been optimized for long read lengths (Carrilho et al.). Therefore, viscosity of the polymer solution could be reduced by lowering the concentration of urea, but this step alone would inevitably reduce the denaturing ability of the polymer solution. Therefore, additional denaturant capacity would have to be added by means of another solvent. After a number of organic solvents commonly used for DNA denaturation were tested, DMSO was selected based on its unexpected performance. The effect of lowering urea concentration on the migration speed of DNA fragments was determined first, followed by optimization of concentrations of both DMSO and urea in the polymer solution for close to 1000 bases read length at 70° C. in a minimum amount of time.

### EXAMPLES

The following examples are presented to illustrate the advantages of the present invention and to assist one of ordinary skill in making and using the same. These examples are not intended in any way otherwise to limit the scope of the disclosure. The contents of all cited references (including literature references, issued patents, published patent applications, and co-pending patent applications) cited throughout this application are hereby expressly incorporated by reference.

### Example 1

### Lowering Urea Concentration in the Separation Matrix

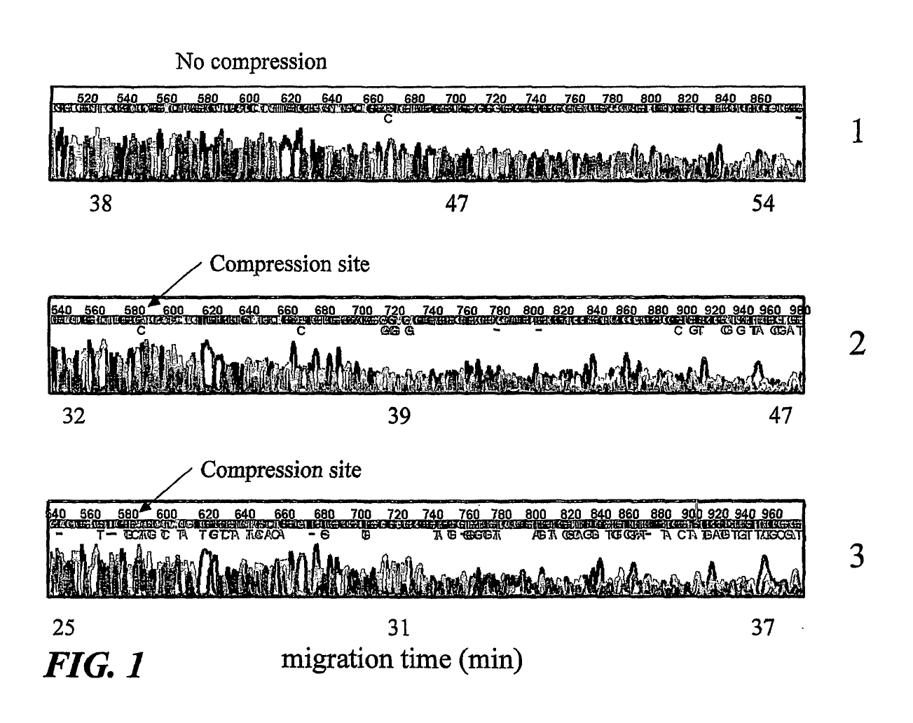

Lowering the urea concentration from 7M down to 3M was tested for its effect on separation speed. Fragments of the electropherograms are presented in FIG. 1. With 6M urea, read length of 930 bases with 98.5% accuracy was generated in roughly one hour, a result similar to that with the separation matrix with 7M urea (FIG. 1A). A further decrease in the urea concentration to 5M resulted in a significant reduction of the read length to 750 bases at 98.5% accuracy. While separation indeed occurred faster at this concentration, requiring less than 48 minutes for the 1000 bases long fragment to pass the detector window, several compressions were not resolved (see below for details about the compression sites), and the resolution of the T-terminated peaks in the late part of the run was substantially lower than that for. other terminations (FIG. 1B). This latter effect could be a result of hydrophobic interactions between dROX (the dye on the T-terminated DNA fragments) and ssDNA (He et al.). Decreasing the urea concentration to 3M resulted in the DNA fragments migrating twice as fast as with 7M urea. However, frequent miscalls made the sequence practically unreadable (FIG. 1C). Thus, lowering urea concentration in the separation matrix indeed resulted in an increase of migration speed of DNA fragments, but this general approach cannot be used alone due to insufficient denaturation of DNA. Two alternative means to improve denaturation were considered—higher column temperature and organic solvent addition.

### Example II

### Thermal Stability of LPA Matrixes at High Temperatures

It is known that at elevated column temperatures, DNA fragments are denatured more effectively. So, it was a question as to whether further temperature increases can possibly improve the denaturing ability of the separation matrix. Previously, it has been found that with LPA matrix solutions, read length decreased rapidly at column temperatures above 70° C. (Zhou et al.). It is also known, however, that urea is prone to thermal decomposition at high temperatures (Nachbaur et al.). Thus, as urea is the principal component in the separation matrix, it is likely to be the principal cause of matrix failure at temperatures above 70° C., and this possibility was investigated.

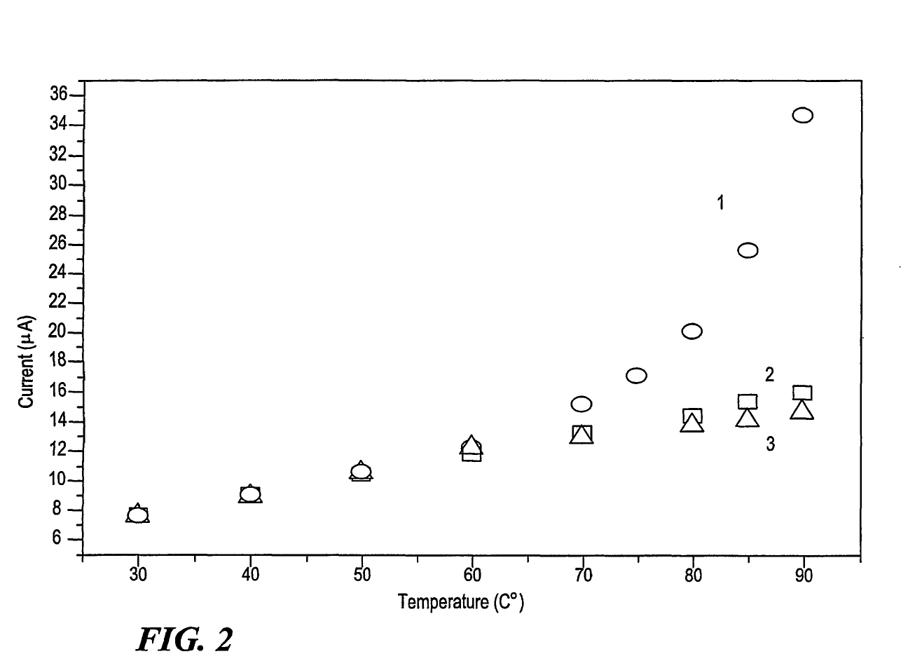

The electric current value and its constancy during separation were chosen as criteria for matrix thermal stability. The electric current at a fixed electric field in the LPA solutions containing either 7M urea or no denaturant at various column temperatures were compared. The current was measured 10 minutes after the start of each run in order to allow sufficient time for its stabilization. The results are presented in FIG. 2. As the column temperature was raised from 30° C. to 70° C., the current increased linearly with both denaturant-free (▪) and the 7M urea-containing (&Ovalhollow;) polymer solutions. However, with the LPA matrix containing 7M urea (&Ovalhollow;), this increase in current became disproportionately high at temperatures above 70° C., and, in addition, at 85-90° C., the current became unstable. When the LPA solution containing only polymer and buffer (no denaturant) (▪) was electrophoresed in the same range of temperatures, the current increased linearly with column temperature, and, importantly, the current was stable at all temperatures up to at least 90° C. Based on the present study, it was concluded that urea decomposition at high temperatures had a concentration-dependent, deleterious effect on separation current. It was also demonstrated that LPA itself can be run at such high temperatures. Optimum temperature for other matrices, e.g., PDMA solutions did not exceed 60° C. (He et al.).

In the process of thermal decomposition, urea can form various products, including ammonia, nitrogen oxides, cyanuric acid, cyanic acid, biuret, and carbon dioxide (see http://www.jtbaker.com/msds/U4725. htm). While it was concluded that significant accumulation of these decomposition products occurred at 130° C. and above (Nachbaur et al.), some significant formation of decomposition products may already occur at column temperatures higher than 70° C. Because of the high electric field of CE, ammonia and other gaseous products may form micro-bubbles and cause instability in the separation current. At the same time, ammonia forms ions in aqueous solutions and, along with other ionic products of decomposition, may increase current in the column to higher values than predicted by Ohm's Law. An increase in the current in turn would cause a non-linear increase in heat generation inside the column, thus, at some point, leading to insufficient heat removal and subsequent loss of efficiency.

DMSO with LPA was then tested. When 5% v/w DMSO was added to the LPA solution, this matrix showed no signs of degradation in the entire tested range of column temperatures up to 90° C. (▾) (see FIG. 2). Therefore, DMSO addition did not change the excellent thermal stability of entanglements in the LPA network. Accordingly, the effect of DMSO concentration in the LPA solution was explored on DNA sequencing.

### Example III

### DNA Sequencing with DMSO-Containing LPA Solutions

DMSO concentrations of 5%, 10%, and 15% v/w were tested with 2.5% w/w 5.6 MDa LPA solutions for DNA sequencing at 70° C. The results, as shown in Table 1, with a comparison to 7M urea, indicate that DNA fragments migrated much faster in DMSO-containing matrices. With increasing DMSO concentration, migration time of DNA fragments was longer, which was expected because of increasing solvent viscosity and more pronounced dielectric friction caused by DMSO (Roy et al.). Even with 15% v/w DMSO in the separation matrix, DNA fragments migrated still substantially faster than with 7M urea. However, at DMSO concentrations higher than 5% v/w, base-calling resulted in shorter read length due to a lower resolution of G- and T-terminated peaks. Since these DNA fragments are labeled with more hydrophobic dyes than the other fragments, this effect could possibly be attributed to an interaction between the dyes and DMSO. Based on the results in Table 1, we concluded that a sufficient concentration of DMSO in the LPA matrix was 5% v/w, as even with this denaturant alone, read length was close to 900 bases with 98.5% accuracy at 70° C. Other data with urea-containing LPA matrices presented in Table 1 will be discussed subsequently.

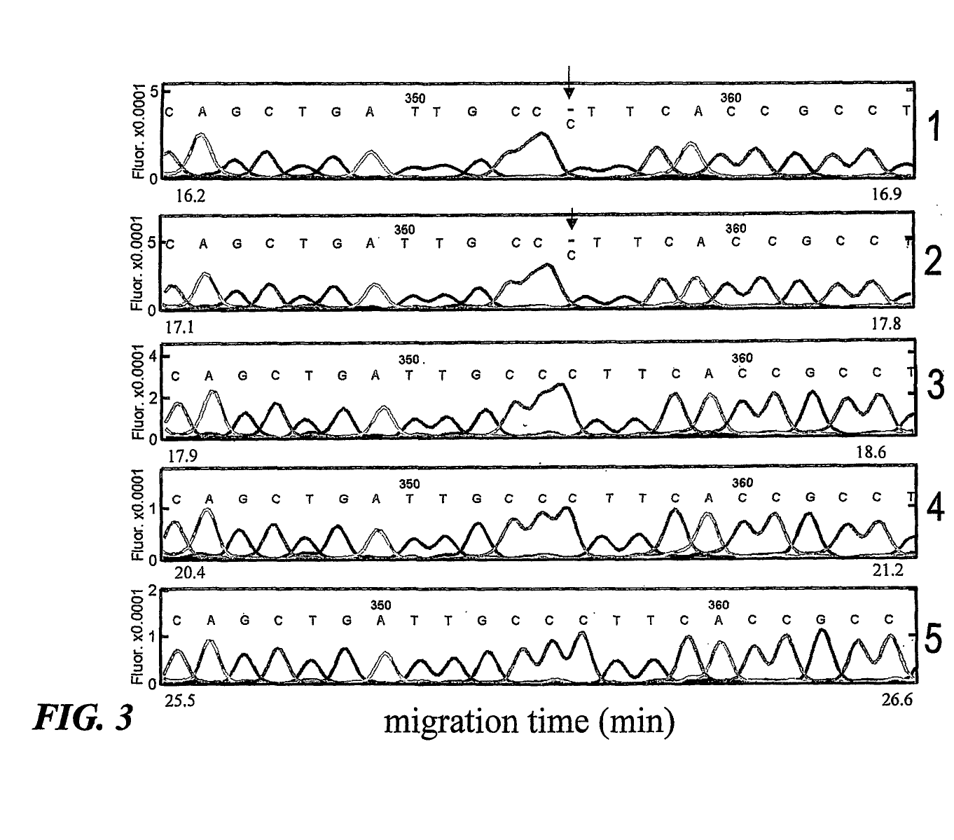

A temperature study of DNA sequencing with 5% v/w DMSO in the 2.5% w/w 5.6. MDa LPA matrix was then performed. At 90° C., the read length of 952 bases with 98.5% accuracy was generated in just 32 minutes, or roughly half of the time needed to complete the run with 7M urea (data not shown). Interestingly, in all runs made with this denaturant at different temperatures, the compression motifs (see FIG. 3A) were not resolved with the column temperature increase, and even at 90° C., separation of these triplets was not sufficient. In addition, taking in account that regular commercial DNA sequencers are not designed to maintain temperature this high, and the capillary coating stability at this temperature may be of a concern, use of 5% v/w DMSO alone as denaturant was not studied further.

TABLE 1
Migration Time for Specific ssDNA Fragments and Read
Length at 70° C. and 200 V/cm in 2.5% (w/w) LPA Solutions Containing
Different Denaturants^{a}

| | | | Maximum | Migration |
|---|---|---|---|---|
| | Migration | Migration | read length | time for |
| | time for | time for | with 98.5% | the highest |
| | base 600 | base 900 | accuracy | base number |
| Denaturant | (min) | (min) | (bases) | (min) |
| 5% v/w DMSO | 24.5 | 33.8 | 882 | 33.3 |
| 10% v/w DMSO | 28.0 | 38.8 | 848 | 37.0 |
| 15% v/w DMSO | 33.4 | N/A | 656 | 36.0 |
| 5% v/w DMSO | 26.0 | 36.3 | 936 | 37.3 |
| + 1 M urea | | | | |
| 5% v/w DMSO | 27.2 | 37.4 | 975 | 39.5 |
| + 2 M urea | | | | |
| 5% v/w DMSO | 31.0 | 42.2 | 950 | 43.3 |
| + 3 M urea | | | | |
| 7 M urea | 38.4 | 52.3 | 1055 | 58.0 |

^{a}See Materials and Methods for other conditions.

### Example IV

### DNA Sequencing with Both DMSO and Urea Containing LPA Matrices

In order to improve resolving compressions while maintaining fast run times at 70° C., we added small amounts of urea to the matrix containing 5% v/w DMSO. As can be seen from Table 1, the addition of only 1 M urea led to increase in the read length by more than 50 bases, while changes in run time, compared to the DMSO matrix, were small. With LPA solution containing 5% v/w DMSO and 2M urea, a read length of 976 bases with 98.5% accuracy was generated in less than 40 minutes. According to the results in Table 1, raising the urea concentration in the LPA matrix over 2M did not lead to increase in the read length.

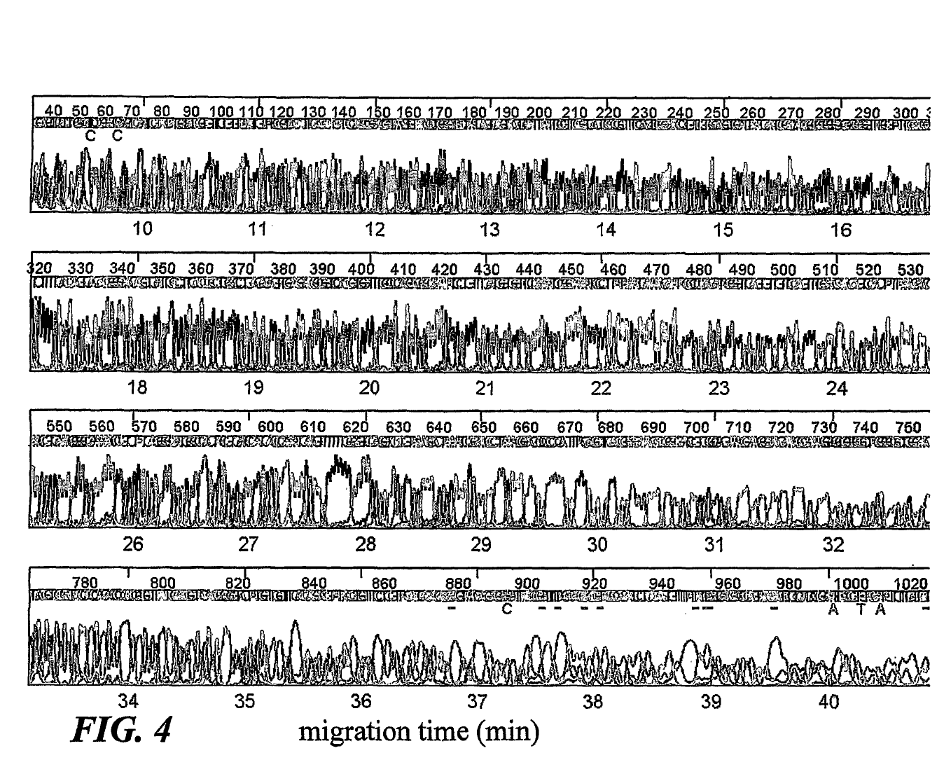

Analyzing electropherograms generated using LPA matrices with 5% v/w DMSO and M13mp18 template, two compression motifs located in the positions 353-355 and 583-585 bases were identified from the first base of the primer recognition site (triplets of the C-terminated peaks) where the base-caller under-called one base. One of these motifs is shown in FIG. 3A. At 1M urea (FIG. 3B), the denaturing ability of the matrix was not sufficient for resolving the compression, while increasing concentration to 2M enhanced the resolution of the adjacent peaks sufficiently that correct base-calling was possible (FIG. 3C). With further increase in the urea concentration to 3M (FIG. 3D), resolution of all three C-terminated peaks was comparable to that of the original matrix with 7M urea (FIG. 3E). This is an important result demonstrating that in sequencing of templates with stronger compressions, higher urea concentrations in the matrix may be required to resolve them. Even in such a case, the resulting sequence still may be generated faster than with an LPA matrix with high concentration of urea. The sequencing run using LPA matrix containing 5% v/w DMSO and 2M urea with ssM13mp18 template is shown in FIG. 4.

The overall throughput of the sequencing process with this matrix (the total number of bases generated in one hour per capillary) was over 30% higher than that with the separation matrix containing 7M urea. This new matrix can be used in both full-length capillary systems and in the integrated microchip devices as well. While with microchips filled with conventional matrices, the separation speed of 500 bases in 30 minutes per channel has been achieved (Medintz et al.), the new matrix almost doubles this throughput using standard capillaries at a price of 10-minute longer separation. The matrix with this alternative denaturant mixture would not require adjusting already optimum concentrations of the polymer and other components of the matrix formulation. Moreover, urea concentration in the matrix can be modified for DNA templates with compressions of various strengths.

Materials and Methods

Instrumentation. The design of the single capillary instrument with laser-induced fluorescence (LIF) was similar to that previously described in (Ruiz-Martinez et al., 1996). The fluorescence emission was collected with a microscope objective (Model 13600, Oriel, Stamford, Conn.), and the spectra of the labeled sequencing fragments were acquired in 16 channels in the range from 500 to 660 nm with a CCD camera (Model NTE/CCD-1340/400-EMB, Roper Scientific, Trenton, N.J.). The laser and other components are the same as reported previously (Ruiz-Martinez et al., 1993; Ruiz-Martinez et al., 1996). The CE columns were fused silica capillaries of 75 μm i.d, 365 μm O.D. (Polymicro Technologies, Inc., Phoenix, Ariz.), covalently coated with polyvinyl alcohol (PVA) (Karger et al.). The capillary was not bent and was placed horizontally in the instrument. The effective capillary length (distance from injection point to the detection window) was 30 cm, with a total length of 45 cm. The sample was injected for 10 sec at constant current of 0.7 μA, and electrophoresis was performed at 200 V/cm.

Chemicals. Acrylamide, N, N, N′, N′-tetramethylethylenediamine (TEMED), ammonium persulfate and urea were purchased from ICN Biomedicals, Inc. (Aurora, Ohio). TRIS, TAPS and EDTA were obtained from Sigma (St. Louis, Mo.), and DMSO was from Aldrich (Milwaukee, Wis.). All reagents were either electrophoresis or analytical grade, and no further purification was performed. Span 80 emulsifier and petroleum special with a boiling range from 180-220° C. were purchased from FLUKA chemicals (Milwaukee, Wis.). Water was deionized with a Milli-Q purification system to 18.2-MΩ grade (Millipore, Worcester, Mass.).

Polymer synthesis and characterization. Linear polyacrylamide (LPA) with molecular mass 5.6 MDa was prepared in powder form using inverse emulsion polymerization, as described previously (Goetzinger et al.). After polymerization, the LPA powder was washed with acetone and vacuum dried. LPA molecular mass was determined by multi-angle laser light scattering (Wyatt).

Preparation of the separation matrices. Polymer solutions containing 2.5% (w/w) LPA (5.6 MDa) and a denaturant were utilized to separate the DNA sequencing reaction products. To prepare a typical mixed solution (20 g), appropriate amounts of dry LPA polymer, denaturant, 10× buffer concentrate (500 mM Tris: 500 mM TAPS: 20 mM EDTA) and water were added in a glass jar and slowly stirred with a magnetic bar. All components were added by weight, except that DMSO was measured volumetrically. The solutions were usually homogenized slowly for two days and then were ready for use. Each polymer solution was replaced from the capillary after a given run using a gas tight syringe, and the voltage was applied for 5 minutes before injection to reduce the current in the matrix solution to a constant value. LPA powder and other dried polymers have almost unlimited shelf life. The working solutions of LPA could be stored in the refrigerator at 4° C. for up to 3 months.

DNA sequencing reactions were performed using standard cycle sequencing chemistry with AmpliTaq-FS and BigDye (−21) M13 universal primers (Applied Biosystems Corp., Foster City, Calif.) on an M13mp18 single-stranded template (New England Biolabs, Beverly, Mass.). ssM13mp18 DNA has become the de facto standard template for use both for research in DNA separation and development of commercial sequencing instrumentation in methods of obtaining long read lengths. While this template does not have many properties of production genomic templates of the “real world,” such as high GC-content, single- and polynucleotide repeats, etc., it is a good model for testing various separation properties of polymer solutions, including an ability to resolve mild compressions, as well as to optimize the polymer solution performance.

The temperature cycling protocol for this sequencing chemistry was made on a PTC200 thermocycler (MJ Research, Inc., Watertown, Mass.), consisting of 15 cycles of 10 s at 95° C., 5 seconds at 50° C. and 1 minute at 70° C., followed by 15 cycles of 10 seconds at 95° C. and 1 minute of 70° C. After completion of the reaction, the samples were heated for 5 minutes at 100° C. in order to inactivate the enzymes prior to the clean-up procedure.

Purification of the reaction products. Sequencing reactions were cleaned using the method described in Ruiz-Martinez et al., Anal. Chem., 70:1516-1527 (1998), incorporated by reference, with minor modifications. Template DNA (M13mp18) was removed using spin columns with a polyethersulfone ultrafiltration membrane, molecular weight cut-off of 300,000 (MWCO 300K, Pall Filtron, Northborough, Mass.), which was pretreated with an 0.005% w/w solution of LPA with a molecular mass 700-1000 kDa. The filtrate was dried under vacuum and dissolved in 50 μL of deionized water. The reconstituted template-free sequencing samples were then desalted using prewashed Centri-Sep 96 (gel filtration) plates (Princeton Separations, Adelphia, N.J.). The desalting procedure was performed twice per sample, after which the sample volume was adjusted to 55 μL.

A 5 μL aliquot of the purified sample was diluted with 20 μL of deionized water prior to injection. The specific injection conditions are described in the figure captions. The purified sequencing samples were stored at −20° C. in deionized water.

Base-calling software. A system for base calling was used in accordance with Salas-Solano, et al., Anal. Chem., 70:3996-4003 (1998) and Miller et al., U.S. Pat. No. 6,236,944 (2001), both of which are incorporated by reference. Data processing began by determining the primer dye spectra from the relatively intense peaks in the data and performing color separation by a least-squares fit to these spectra. The electropherogram was divided into sections containing 20-40 bases, and the fifth percentile value among the amplitudes of all data points in each section was computed. This calculation established the background at the center of each section, and elsewhere it was derived by linear interpolation. After background subtraction, the starting point of the sequence-containing region was determined by locating the primer peak and examining the time interval for the beginning of relatively uniform peak heights. The end point was designed as a migration time shortly before the position at which oriented reputation caused the elution of the remaining DNA as a single peak, which was detected by a dramatic increase in the standard deviation of the signal. If no such terminating peak was found, the end point was the end of the electropherogram. Dye mobility shifts and average peak heights were computed throughout the sequence-containing region, and a set of empirical rules was employed to find peak boundaries and estimate the number of bases in each peak. The optimum performance on DMSO-containing matrices required minor changes to the parameters of some rules developed for matrices containing 7 M urea, due to higher separation speed, lower peak resolution, etc. Sequencing data (read length, migration time, etc.) obtained from the base-caller were processed with Origin 6.0 software (Microcal, Northampton, Mass.). For the present invention, the software was modified to perform base-calling at a peak resolution as low as 0.24.

### USE

Based on the foregoing, a new formulation of an uncrosslinked polymer separation matrix containing DMSO, preferably both DMSO and urea, and most preferably 5% v/w DMSO and 2-3M urea was developed for rapid nucleic acid sequencing by capillary array and/or microchip electrophoresis, giving results with read lengths close to 1000 bases. This matrix solution combines the high resolving power of, for example, LPA solutions previously optimized for long read lengths but with the improvement of increased separation speed. The total throughput of DNA sequencing with this matrix may be over 30% higher than that with the same LPA matrix containing 7M urea. Compared to current commercial LPA separation matrices, the throughput of sequencing may be even higher due to larger difference in the separation speed. This new matrix optimum temperature is 70° C., which is identical to the matrices containing the same LPA and 7M urea, and its utilization is possible in some of the commercially available DNA sequencers without sequencer modification. Other LPA matrices could also be enhanced for faster separation at their optimum temperatures by replacing high urea concentration with the mixture of 2M urea and 5% v/w DMSO without the need of reoptimization. For sequencing DNA templates with stronger compressions, the denaturing ability of this mixture may be readily increased by raising the urea concentration to 3-4M. Even in this case, separation was still faster than that observed with 7M urea in the matrix. For resolution of very strong compressions, a urea concentration up to 7M may be used in conjunction with DMSO. In this case, however, separation speed will be sacrificed for improved resolution.

Even more generally, the separation matrix of the invention is useful for electrophoretic separation of nucleic acids in any type of analytical method that would be useful for, e.g., genotyping, SNP profiling, scoring, etc. Such methods include, inter alia, single strand conformational polymorphism (SSCP) determination; constant denaturant/capillary electrophoresis (CD/CE); and restriction fragment length polymorphism (RFLP) analysis. Assay conditions may vary with each method of analysis. However, in accordance with the invention, the separation matrix must include DMSO as a denaturant, as described above. DMSO may be used in combination with urea, as described above. For example, approximately 5% v/w DMSO alone is considered gentle enough to denature a three-dimensional structure, such as a double stranded DNA, at room temperature. While the temperature of the assay condition is a function of the polymer used, DMSO is also found to be stable at elevated temperatures, which allows for reduced separation time and increased resolution of compressions as well as improved selectivity (i.e., extent of difference in electrophoretic mobilities) for long nucleic acid fragments, due to shifting of the onset of biased reptation to higher base numbers (Fang et al.).

### Referemces

Albarghouthi, M. N. and Barron, A. E., Electrophoresis, 2000, 21, 4096-4111.

Carrilho et al., Anal. Chem., 1996, 68:3305-3313.

Chakrabarti, R. and Schutt, C. E., Nucleic Acids Res., 2001, 29, 2377-2381.

Chang, H. T. and Yeung, E. S., Electrophoresis, 1995, 16, 2069-2073.

Choi, J. S., Kim, J. S., Joe, C. O., Kim, S., Ha, K. S. and Park, Y. M., Exp. Mol. Med., 1999, 31, 20-24.

Dolnik, V., J Biochem. Biophys. Methods, 1999, 41, 103-119.

Dovichi, N. J., Electrophoresis, 1997, 18, 2393-2399.

Escara, J. F. and Hutton, J. R., Biopolymers, 1980, 19, 1315-1327.

Fang, Y., Zhang, J. Z., Hou, J. Y., Lu, H. and Dovichi, N. J., Electrophoresis, 1996, 17, 1436-1442.

Flanagan, J. H., Jr., Owens, C. V., Romero, S. E., Waddell, E., Kahn, S. H., Hammer, R. P., and Soper, S. A., Anal. Chem., 1998, 70, 2676-2684.

Goetzinger, W., Kotler, L., Carrilho, E., Ruiz-Martinez, M. C., Salas-Solano, and Karger, B. L., Electrophoresis, 1998, 19, 242-248.

He, H., Buchholz, B., Kotler, L., Miller, A. W., Barron, A. E. and Karger, B. L., Electrophoresis, submitted.

Head, D. L. and McCarty, C. G., Tetrahedron Lett., 1973, 16, 1405-1408.

Heath, K. E., Humphries, S. E., Middleton-Price, H. and Boxer, M., Eur. J. Hum. Genet., 2001, 9, 244-252.

Heller, C., Electrophoresis, 2001, 22, 629-643.

http://www.jtbaker.com/msds/D7120. htm.

http://www.jtbaker.com/msds/U4725. htm.

Hui-Shieh, C. and Hedayati, A., US Pat. 5,961,801, 1999.

Hutton, J. R., Nucleic Acids Res., 1977, 4, 3537-3555.

Jung, M., Muche, J. M., Lukowsky, A., Jung, K. and Loening, S. A., Anal. Biochem., 2001, 289, 292-295.

Karger, B. and Goetzinger, W., US Pat. 5,840,388, 1998.

Khmelnitsky, Y. L., Mozhaev, V. V., Belova, A. B., Sergeeva, M. V. and Martinek, K., Eur. J. Biochem., 1991, 198, 31-41.

Kleparnik, K., Foret, F., Berka, J., Goetzinger, W., Miller, A. W. and Karger, B. L., Electrophoresis, 1996, 17, 1860-1866.

Konrad, K. D. and Pentoney, S. L., Jr., Electrophoresis, 1993, 14, 502-508.

Lander, E. S., et al., Nature, 2001, 409, 860-921.

Li, L. and McGown, L. B., Fresenius J. Anal. Chem., 2001, 369, 267-272.

Medintz, I. L., Paegel, B. M. and Mathies, R. A., J. Chromatogr. A, 2001, 924, 265-270.

Miller, A. W. and Karger, B. L., US Pat. 6,236,944, 2001.

Nachbaur, E., Baumgartner, E. and Schober, J., Proc. Eur. Symp. Therm.Anal., 1981, 417-421.

Roy, K. I. and Lucy, C. A., HPCE 2001, Boston, 2001, 65.

Ruiz-Martinez, M. C., Berka, J., Belenkii, A., Foret, F., Miller, A. W. and Karger, B. L., Anal. Chem., 1993, 65, 2851-2858.

Ruiz-Martinez, M. C., Carrilho, E., Berka, J., Kieleczawa, J., Miller, A. W., Foret, F., Carson, S. and Karger, B. L., Biotechniques, 1996, 20, 1058-1064, 1066-1059.

Ruiz-Martinez, M. C., Salas-Solano, O., Carrilho, E., Kotler, L. and Karger, B. L., Anal. Chem., 1998, 70, 1516-1527.

Salas-Solano, O., Carrilho, E., Kotler, L., Miller, A. W., Goetzinger, W., Sosic, Z. and Karger, B. L., Anal. Chem., 1998, 70, 3996-4003.

Schmitz, G., Aslanidis, C. and Lackner, K. J., Clin. Chim. Acta, 2001, 308, 43-53.

Seto, D., Seto, J., Deshpande, P. and Hood, L., DNA Seq., 1995, 5, 131-140.

Sun, Y., Hegamyer, G. and° C.olburn, N. H., Biotechniques, 1993, 15, 372-374.

Tan, H. and Yeung, E. S., Electrophoresis, 1997, 18, 2893-2900.

Venter, J. C., et al., Science, 2001, 291, 1304-1351.

Weitzman, J., Trends Mol. Med., 2001, 7, 287.

Wyatt, P. J., Anal. Chim. Acta, 1993, 272, 1-40.

Yoshihiro, A., Japan Pat. JP9145673A2, 1997.

Zhang, J., Fang, Y., Hou, J. Y., Ren, H. J., Jiang, R., Roos, P. and Dovichi, N. J., Anal. Chem., 1995, 67, 4589-4593.

Zhang, W. and Deisseroth, A. B., Methods Mol. Biol., 1996, 65, 137-147.

Zhou, H., Miller, A. W., Sosic, Z., Buchholz, B., Barron, A. E., Kotler, L. and Karger, B. L., Anal. Chem., 2000, 72, 1045-1052.

Zimmer, C. and Luck, G., Biochim. Biophys. Acta, 1972, 287, 376-385.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A method of high throughput nucleic acid sequencing, said method comprising the steps of:
a) providing a nucleic acid sample to be sequenced;
b) carrying out nucleic acid sequencing reactions on said sample, thereby generating a product;
c) injecting an aliquot of said product into a separation device, said device comprising an uncrosslinked polymer matrix solution and a denaturant comprising dimethyl sulfoxide;
d) separating said product into component parts using said device; and
e) determining the sequence of nucleotides in said nucleic acid sample from the results of said separation step.

2. The method of claim 1, wherein said device is a capillary electrophoretic device.

3. The method of claim 2, wherein said device comprises a capillary column.

4. The method of claim 3, wherein said device comprises a capillary array.

5. The method of claim 2, wherein said device comprises a microscale liquid handling substrate having one or more channels integrally formed therein for conducting a liquid sample in said substrate.

6. The method of claim 1, wherein said uncrosslinked polymer is selected from the group consisting of linear polyacrylamide, poly(ethylene oxide), hydroxyethyl cellulose, poly(dimethylacrylamide) and poly(vinylpyrrolidone).

7. The method of claim 1, wherein said denaturant further comprises urea.

8. The method of claim 1, wherein said dimethyl sulfoxide is at a concentration of 1% to 25% v/w.

9. The method of claim 1, wherein said dimethyl sulfoxide is at a concentration of 5% v/w.

10. The method of claim 7, wherein said urea is at a concentration of 0.5M to 7M.

11. The method of claim 7, wherein said urea is at a concentration of 2-3M.

12. The method of claim 7, wherein, in step c), said denaturant comprises 5% v/w dimethyl sulfoxide and 2M urea.

13. The method of claim 1, wherein said separation step d) is conducted at a temperature of 25° C. to 50° C.

14. The method of claim 1, wherein said separation step d) is conducted at a temperature of 50° C. or higher.

15. The method of claim 1, wherein said separation step d) is conducted at a temperature of 60° C. or higher.

16. The method of claim 1, wherein said separation step d) is conducted at a temperature of 70° C. or higher.

17. The method of claim 1, wherein said separation step d) is conducted at a temperature in the range of 70° C. to 80° C.

18. The method of claim 1, wherein said separation step d) is conducted at a temperature in the range of 80° C. to 90° C.

19. A method of high throughput nucleic acid sequencing, said method comprising the steps of:
a) providing a nucleic acid sample for sequencing;
b) carrying out nucleic acid sequencing reactions on said sample, thereby generating a product;
c) injecting an aliquot of said product into a separation device, said device comprising a linear polyacrylamide polymer matrix solution and a denaturant comprising 1% to 25% v/w dimethyl sulfoxide;
d) separating said product into component parts using said device at a temperature in the range of 60° C. to 80° C.; and
e) determining the sequence of nucleotides in said nucleic acid sample from the results of said separation step.

20. The method of claim 19, wherein said device is a capillary electrophoretic device.

21. The method of claim 20, wherein said device comprises a capillary column.

22. The method of claim 21, wherein said device comprises a capillary array.

23. The method of claim 20, wherein said device comprises a microscale liquid handling substrate having one or more channels integrally formed therein for conducting a liquid sample in said substrate.

24. The method of claim 19, wherein said denaturant further comprises urea.

25. The method of claim 24, wherein said urea is at a concentration of 2-3M.

26. The method of claim 24, wherein said denaturant is 2% v/w dimethyl sulfoxide and 2M urea, and wherein said temperature is 70° C.

27. A separation matrix for nucleic acid electrophoretic analysis comprising:
an uncrosslinked polymer matrix solution; and
a denaturant comprising dimethyl sulfoxide.

28. The separation matrix of claim 27, wherein said denaturant further comprises urea.

29. The separation matrix of claim 28, wherein said denaturant comprises 1% to 25% v/w dimethyl sulfoxide and 0.5M to 7M urea.

30. The separation matrix of claim 27, wherein said uncrosslinked polymer is selected from the group consisting of linear polyacrylamide, poly(ethylene oxide), hydroxyethyl cellulose, poly(dimethylacrylamide) and poly(vinylpyrrolidone).

31. A method of electrophoretic analysis of nucleic acids, said method comprising the steps of:
a) providing a nucleic acid sample to be analyzed;
b) carrying out steps of a nucleic acid analytical method that produce a product to be separated into component parts;
c) injecting an aliquot of said product into a separation device, said device comprising a uncrosslinked polymer matrix solution and a denaturant comprising dimethyl sulfoxide;
d) separating said product into component parts using said device; and
e) determining the final results of said analytical method on said nucleic acid sample from the results of said separation step.

32. The method of claim 31, wherein said uncrosslinked polymer is selected from the group consisting of linear polyacrylamide, poly(ethylene oxide), hydroxyethyl cellulose, poly(dimethylacrylamide) and poly(vinylpyrrolidone).

33. The method of claim 31, wherein said device is a capillary electrophoretic device.

34. The method of claim 33, wherein said device comprises a capillary column.

35. The method of claim 34, wherein said device comprises a capillary array.

36. The method of claim 33, wherein said device comprises a microscale liquid handling substrate having one or more channels integrally formed therein for conducting a liquid sample in said substrate.

37. The method of claim 31, wherein said denaturant further comprises urea.

38. The method of claim 31, wherein said analytical method is selected from the group consisting of single strand conformational polymorphism (SSCP) determination, constant denaturant/capillary electrophoresis (CD/CE) and restriction fragment length polymorphism (RFLP) analysis.

39. The method of claim 31, wherein said dimethyl sulfoxide is at a concentration of 1% to 25% v/w.

40. The method of claim 31, wherein said dimethyl sulfoxide is at a concentration of 5% v/w.

41. The method of claim 37, wherein said urea is at a concentration of 0.5M to 7M.

42. The method of claim 37, wherein said urea is at a concentration of 2-3M.

43. The method of claim 37, wherein said denaturant comprises 5% v/w dimethyl sulfoxide and 2M urea.

44. The method of claim 31, wherein said separation step d) is conducted at a temperature of 30° C. or higher.

45. The method of claim 31, wherein said separation step d) is conducted at a temperature of 40° C. or higher.

46. The method of claim 31, wherein said separation step d) is conducted at a temperature of 50° C. or higher.

47. The method of claim 31, wherein said separation step d) is conducted at a temperature of 60° C. or higher.

48. The method of claim 31, wherein said separation step d) is conducted at a temperature of 70° C. or higher.

49. The method of claim 31, wherein said separation step d) is conducted at a temperature in the range of 70° C. to 80° C.

50. The method of claim 31, wherein said separation step d) is conducted at a temperature in the range of 80° C. to 90° C.
